(19) United States
(12) Patent Application Publication
(10) Pub. No.: US 2006/0228308 A1
(43) Pub. Date: Oct. 12, 2006

(54) Oral health care drink and method for reducing malodors

(76) Inventors: Barry W. Cummins, Ft. Pierce, FL (US); David H. Creasey, Vero Beach, FL (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(21) Appl. No.: 11/017,894

(22) Filed: Dec. 21, 2004

Related U.S. Application Data
(60) Provisional application No. 60/547,991, filed on Feb. 26, 2004.

(51) Int. Cl. A61K 8/97; A61K 36/906; A61K 33/34; A61K 33/38; A61K 33/32

(52) U.S. Cl. 424/58; 424/741; 424/618; 424/638; 424/641

(57) ABSTRACT

An oral care composition and method of using are provided for treating and preventing malodors or disease conditions of the oral cavity in warm-blooded animals, including humans. When applying an oral care effective amount of the oral care composition of the present invention to the mucosal tissue of the oral cavity, the gingival tissues of the oral cavity, and/or surface of the teeth. The application can be as a liquid for bathing, rinsing or gargling, or lather generated by brushing the teeth for a sufficient time. After rinsing, gargling or brushing, the composition of the present invention should be swallowed to achieve effective reduction of various organisms, diseases and malodors. An oral care effective amount can also prevent and heal mouth sores and canker sores.

This invention claims the benefit of priority based on the U.S. Provisional Application Ser. No. 60/547,991 filed Feb. 26, 2004.

### FIELD OF THE INVENTION

This invention relates to an oral health care composition and in particular to a composition, a method of making and using the composition as an oral health care drink and for the reduction of oral cavity, food and drink malodor.

### BACKGROUND AND PRIOR ART

Oral health care has been and will continue to be a concern in the human race and in many other warm-blooded animals, notably horses, dogs, cats and the like. For human beings, oral malodor, also known as “bad breath” or halitosis can be embarrassing for people who are experiencing it. In business and professional careers, bad breath can determine whether a deal is completed or lost. In personal relationships, it can mean a lot to comfort and closeness of both parties. The breath generally is one of the first impressions a person leaves with another, so it is an extremely important component of good grooming. As the world population increases, people and other warm-blooded animals live in more crowded and limited spaces; malodors from any source become a concern; clearly there is more to warm-blooded animal malodor than meets the nose.

Many factors can be involved in bad breath, including gum disease, tooth decay, heavy metal buildup, sinus infection, nose and throat infection, improper diet, dry mouth, gastric problems, constipation, smoking, diabetes, indigestion, postnasal drip, stress, coffee, foods containing onions, garlic, cabbage, various spices, inadequate protein digestion and the like. The preceding list of factors is far from complete. Unfortunately, most people have bad breath at one time or another. The composition of the present invention is effective in reducing oral cavity malodor caused by many of the factors named above.

Folk remedies for bad breath include, chewing parsley, mint leaves, cloves, guava peels, and eggshells, in order to mask the malodor. Mouthwashes have been used as a chemical approach to combat oral malodor. Most commercial mouth rinses/mouthwashes only mask odors, providing little antiseptic function. The existing to commercial products generally contain flavoring, dye and alcohol as the antiseptic substance; the effects are usually not long-lasting. The microbes survive antiseptic attacks by being protected under thick layers of plaque and mucus. So, while the antiseptic mouthwash may kill bacteria that cause bad breath, the bacteria soon return in greater force. In addition, if the antiseptic agent is an alcohol, such as ethanol, the most prevalent problem with ethanol is that it can dry the oral tissues. This condition in itself can actually induce oral malodor. Alcohol is also non-selective in killing bacteria in the mouth, killing both good and bad bacteria. So, it would be desirable to have a long-lasting, alcohol-free composition for treating oral malodor.

The following patents describe the use of copper salts, copper sulfate, ammonium sulfate and sulfuiric acid in disinfectants and treatments for oral cavity conditions. U.S. Pat. No. 5,840,322 to Weiss et al. uses copper ions and copper salts as an anti-plaque agent. U.S. Pat. No. 5,879,663 to Nakabayashi et al. discloses a composition for dentin hypersensitivity containing sulfuric acid, copper sulfate and ammonium compounds. U.S. Pat. No. 5,997,911 to Brinton et al. use copper sulfate, sulfuric acid and ammonium salts solubilized in drinking water for turkeys and swine as an antimicrobial agent. U.S. Pat. No. 6,689,342 B1 to Pan et al. discloses an oral composition containing sulfuric acid, stabilizers, ammonium salts, copper salts in an organic compound based mixture to treat a variety of oral conditions, including malodor.

With regard to health problems and improper diet, the oral health of a person can result in canker sores and mouth sores that can be very painful and uncomfortable, in addition to creating an unsightly appearance. There is a need for a composition that can relieve the suffering caused by sores in the oral cavity.

The present invention provides an inorganic antimicrobial composition that is effective for treating canker sores and mouth sores and, also, effectively controls oral malodor caused by a multitude of factors. More specifically, the present invention controls malodors caused by salivary juices acting on particles of leftover food trapped in the mouth and malodors from certain foods such as onion and garlic, as well as tobacco and alcohol that produce odors that linger in the mouth despite brushing.

In U.S. Pat. Nos. 5,989,595 and 6,242,011 B1 to Cummins, an acidic composition of matter is disclosed that is useful for destroying microorganisms that spoil food, such as fish. The composition of matter, patented by Cummins, is also useful for skin treatment of melanoma and the treatment of other bacteria, and serves as the precursor for the novel composition and method for reducing oral, food and drink malodor disclosed herein.

### SUMMARY OF THE INVENTION

The first objective of the present invention is to provide an oral health care composition that controls food malodors.

The second objective of the present invention is to provide an oral health care composition that controls oral malodor from ingested garlic.

The third objective of the present invention is to provide an oral health care composition that controls oral malodor from ingested onion.

The fourth objective of the present invention is to provide an oral health care composition that controls oral malodor from smoking tobacco.

The fifth objective of the present invention is to provide an oral health care composition that effectively treats ulcerated conditions in the oral cavity, such as canker sores.

The sixth objective of the present invention is to provide an oral health care composition that effectively treats ulcerated conditions in the oral cavity, such as mouth sores.

The seventh objective of the present invention is to provide a composition that reduces malodor from handling onions and garlic during food preparation.

The eighth objective of the present invention is to provide a composition that reduces oral cavity malodor from smoking tobacco.

A preferred oral care composition is provided when an oral care effective amount of PHB0020 with uniformly suspended metallic ions is mixed with an edible inorganic acid, at least one alkali-metal benzoate compound, ginger, polyglycol, sorbitol, pyridoxine (vitamin B6) and water to form a solution for use in the oral cavity of warm-blooded animals.

A more preferred oral care composition contains a sweetener, such as xylitol and metallic ions, such as copper, silver and zinc; most preferably copper ions.

The edible inorganic acid in the composition is at least one of citric acid, ascorbic acid and phosphoric acid; more preferably a combination of citric acid and ascorbic acid.

The alkali-metal benzoate compound used in the oral care composition can be sodium benzoate, potassium benzoate and calcium benzoate, more preferably, a combination of sodium benzoate and potassium benzoate.

The preferred method of preparing an oral care composition that is useful for reducing malodors from food, tobacco and beverage products includes mixing PHB0020 containing metallic ions with deionized water; adding an edible inorganic acid, adding at least one alkali benzoate compound, ginger, poly glycol, sorbitol, xylitol, pyridoxine (vitamin B6), and stirring until completely blended and substantially all metallic ions are uniformly suspended.

The preferred metallic ions are copper, silver and zinc, more preferably copper ions.

The preferred edible inorganic acid is citric acid, ascorbic acid or phosphoric acid, more preferably, a combination of citric acid and ascorbic acid.

The preferred alkali-metal benzoate compound is sodium benzoate, potassium benzoate and calcium benzoate, more preferably a combination of sodium benzoate and potassium benzoate.

A preferred method for treating or preventing ulcerated conditions of the oral cavity in warm-blooded animals, including humans includes applying an oral care effective amount of the composition of the present invention the oral cavity, preferably the composition contains a sweetener, such as xylitol. The composition can be in the form of a paste, gels, mouth sprays and lozenges that can be liquefied in the mouth, rinsed and swallowed. The ulcerated conditions can include, but are not limited to, canker sores and mouth sores.

A preferred method for treating or preventing oral cavity malodors resulting from the ingestion or inhalation of substances selected from the group consisting of onion, garlic, coffee, and tobacco comprising applying an oral care effective amount of the composition of the present invention to the oral cavity, which includes the mucosal tissue, gingival tissues and/or surface of the teeth. The composition of the present invention can be in the form of a paste, gel, mouth spray and lozenge that can be liquefied, rinsed and swallowed.

The composition of the present invention can be used to remove the malodor of peeled, odoriferous raw vegetables by applying a dilute solution of said composition to a surface that is in contact with peeled, odoriferous raw vegetables. The surfaces from which the malodors can be removed include, but are not limited to, hands, counter tops, cutlery, clothing and cutting boards.

Further objects and advantages of this invention will be apparent from the following detailed description of a presently preferred embodiment, which is illustrated schematically in the accompanying drawings.

### BRIEF DESCRIPTION OF THE FIGURES

FIG. 1 is a graph showing the effect of PHB0020 on pathogenic and spoilage bacterial isolates exposed for 2 minutes.

FIG. 2 is a graph showing the logarithm of reductions in bacterial colony levels.

FIG. 3 is a graph of the results of a garlic study using the composition of the present invention (Close Call) and a placebo.

FIG. 4 is a graph of the results of an onion study using the composition of the present invention (Close Call) and a placebo.

FIG. 5 is a graph of the results of a coffee study using the composition of the present invention (Close Call) and a placebo.

FIG. 6 is a graph of the results of a tobacco study using the composition of the present invention (Close Call) and a placebo.

### DESCRIPTION OF THE PREFERRED EMBODIMENT

Before explaining the disclosed embodiment of the present invention in detail, it is to be understood that the invention is not limited in its application to the details of the particular arrangement shown since the invention is capable of other embodiments. Also, the terminology used herein is for the purpose of description and not of limitation. It would be useful to discuss the meanings of some words used herein and their applications before discussing the composition of matter and method of using and making the same: PHB0020—Copper sulfate pentahydrate and/or other forms of copper ions, and silver sulfate and/or other forms of silver ions added to pHarlo for the antimicrobial, anti-bacterial additive of the present invention.
pHarlo—composition of matter claimed in U.S. Pat. Nos. 5,989,595 and 6,242,001 B1 to Cummins and incorporated herein by reference and more completely described below.
Salmonella—Salmonella typhimurium, a pathogen
Listeria—Listeria monocytogenes, a pathogen
Staph—Staphylococcus aureus, a pathogen
E-coli—Escherichia coli, indicator bacteria
Pseudomonas—Pseudomonas fluorescens, spoilage bacteria
Shewanella—Shewanella putrefaciens, spoilage bacteria
malodor—undesirable scent
Close Call—oral care and odor reducing composition of the present invention that is formulated with and without a sweetener, such as xylitol.

The acidic composition of matter and method of making are similar to that described in U.S. Pat. Nos. 5,989,595 and 6,242,011 B1 to Cummins and are incorporated herein by reference.

First, a pressurized vessel is selected that includes a cooling jacket and no electrode attachments; however, the preferred pressurized vessel is fitted with two electrodes, a cathode and anode, to provide a direct current (DC) voltage one (1) foot above the bottom of the container. The electrodes are spaced approximately three (3) feet apart.

The processing steps of the present invention comprise combining sulfuric acid with purity in a range from approximately 94% to approximately 99.9%, in a 1 to 2 volume ratio with distilled water and ammonium sulfate in a ratio of 2.77 pounds of ammonium sulfate per gallon of distilled water to provide mixture (I). The mixture (1) is combined in a pressurized vessel having preferably two strategically placed electrodes, a cathode and anode. During the addition of ammonium sulfate, a direct current (DC) voltage is applied to the mixture. The voltage is applied in a range from approximately one (1) amp to approximately 100 amps, preferably between approximately 1 amp and approximately 5 amps. The mixture is then heated under pressure in a range of from approximately 1 pound per square inch (psi) to approximately 15 psi above atmospheric pressure. Heating of the mixture is in a range of from approximately 200° Fahrenheit (F.) to approximately 1200° F., preferably from approximately 800° F. to approximately 900° F. for approximately 30 minutes. With the application of heat and pressure as specified above, it is understood by persons skilled in the art, that a judicious selection of temperature, time and pressure is required and should be adjusted to maintain a safe chemical reaction.

After cooling the mixture, a stabilizer is added. The stabilizer is a portion of mixture (I) prior to heating in the pressure vessel. The quantity of stabilizer used is approximately 10 weight percent of the total weight of mixture (I). The resulting acidic composition is useful for destroying microorganisms, having a pH of negative 3 (−3). The inventive step of the present invention requires the addition of compounds containing metallic ions for the extensive antimicrobial properties discussed herein. The following physical and chemical properties are observed when undiluted.

pH=−3 which was determined by a non acidified hydrogen proton count with the data corrected for any electrode type errors, and was performed by EFE&H analytical services, an EPA (Environmental Protection Agency) approved laboratory.

stability of metallic ions in solution: from approximately 0 pH up to approximately 9 pH

stability of metallic ions with temperature: from approximately 32° F. to the point of vaporization or approximately 212° F.

Various other compounds with metallic ions may be substituted for copper sulfate pentahydrate. The following metal salts are suitable substitutes:

Copper sulfate, copper glutamate, zinc oxide, zinc glutamate, magnesium glutamate, magnesium sulfate, silver sulfate, silver oxide, and combinations thereof.

Referring now to the composition of pHarlo Blue 0020, hereinafter referred to as PHB0020, it is an antimicrobial, anti-bacterial agent, which has a formulation that is generally recognized as safe (GRAS) by the US Food and Drug Administration. The composition is listed below:

| Ingredient | Percentage |
|---|---|
| Copper Sulfate | 16.4 |
| Pentahydrate | |
| Sulfuric Acid | 9.9 |
| (processing aid) | |
| Ammonium | 2.2 |
| sulfate | |
| Distilled water | 71.5 |

The ingredients form a concentrate, which is combined in small amounts of less than 0.10 milliliters (ml) with 1 gallon of water to make PHB0200.

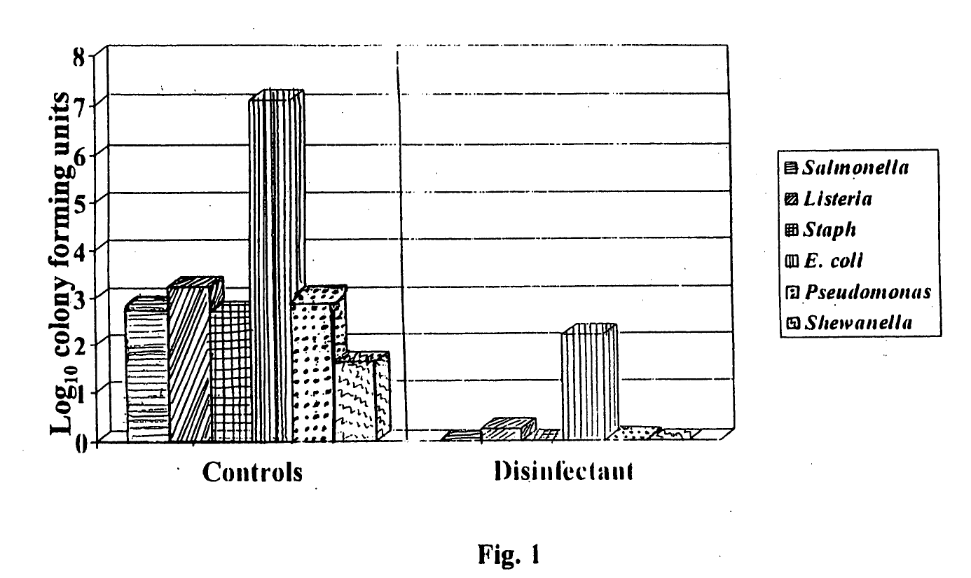

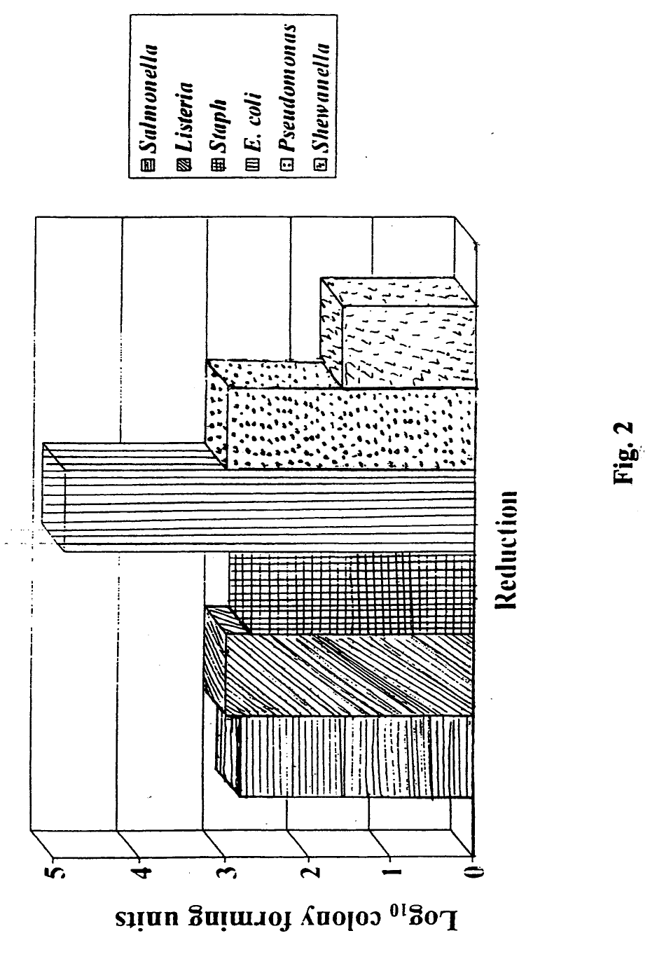

Example 1, Table I, FIG. 1, and FIG. 2 provide greater detail on the use and effectiveness of PHB0020 as an antimicrobial agent.

### EXAMPLE 1

In processing plants for poultry and animal products, it is customary to use various water treatment processes, such as a scalding tank, spray bath, final rinse and chill water tank. The scalding tank is used to dip poultry prior to the removal of feathers; other animals are dipped to remove the outer coating of fur or hair. The scalding process permits cross contamination and spread of pathogens. It is important for the safety of the human food supply to provide an additive that can be used in water treatments to inhibit the growth and spread of pathogens and deleterious bacteria. The ideal additive would not evaporate at boiling point temperatures, would not be destroyed by high temperatures and would not be bound by organic material, such as blood and feces and rendered useless.

The effect of PHB0020 on pathogenic, indicator, and spoilage populations of bacteria associated with broiler chicken carcasses in a poultry scald water application is determined in one embodiment of the present invention.

First, scalder water was collected from the overflow or entrance end of a commercial poultry scalder. The water is sterilized or autoclaved to eliminate all populations of bacteria and bacterial spores to avoid interference during the study. The autoclaved scalder water is evaluated chemically and compared to raw scalder water to ensure that the organic material demand in raw and autoclaved scalder water is similar.

Next, sets of test tubes are prepared by adding 9 milliliters (ml) of sterilized scalder water to sterile polystyrene test tubes. One set is prepared as controls by adding 9 ml of sterilized scalder water to tubes. One set is prepared by adding 9 ml of sterilized scalder water and PHB0020 (the disinfectant) until the pH of 2.2 is achieved.

Each bacterium is exposed, one at a time, to the sterilized scalder water with PHB0020 sanitizer for approximately 2 minutes at approximately 130° F. (55° C.) to mimic scalding.

After the exposure period, one ml of the suspension was enumerated using the aerobic plate count method by pour plating and incubating at approximately 95° F. (35° C.) for 48 hours.

Table I below records microbial growth results in a scalder water project wherein sterilized water was heated to scalding temperatures of in a range of from approximately 120° F. (49° C.) to approximately 140° F. (60° C.), preferably to a temperature of approximately 130° F. (55° C.). Various concentrations of PHB0020 are added in a range between approximately 0.4 parts per million (ppm) to approximately 0.8 ppm, preferably at approximately 0.6 ppm and colonies of pathogens, indicator bacteria and spoilage bacteria are exposed to the treated scalder water.

TABLE I
Scalder Water Project

| | | | |
|---|---|---|---|
| Sample | Colonies forming | | Growth after Exposure |
| No.: | Bacteria Units | Log of Reduction | to Treated Scalder Water |

Bacteria: Salmonella typhimurium
Control

| | | | |
|---|---|---|---|
| 1 | 430 | 2.633468 | negative (no growth) |
| 2 | 880 | 2.944483 | negative |
| 3 | 970 | 2.986772 | negative |
| 4 | 450 | 2.653213 | negative |
| 5 | 620 | 2.792392 | negative |
| 6 | 700 | 2.845098 | negative |
| 7 | 1140 | 3.056905 | negative |
| 8 | 620 | 2.792392 | negative |
| 9 | 580 | 2.763428 | negative |

Bacteria: Staphylococcus aureus
Control

| | | | |
|---|---|---|---|
| 1 | 530 | 2.724276 | negative (no growth) |
| 2 | 550 | 2.740363 | one (1) colony growing |
| 3 | 580 | 2.763428 | negative |
| 4 | 500 | 2.698970 | negative |
| 5 | 540 | 2.732394 | negative |
| 6 | 420 | 2.623249 | negative |
| 7 | 530 | 2.724276 | negative |
| 8 | 480 | 2.681241 | one (1) colony growing |
| 9 | 470 | 2.672098 | negative |

Bacteria: Pseudomonas fluorescens
Control

| | | | |
|---|---|---|---|
| 1 | 540 | 2.73234 | negative |
| 2 | 880 | 2.944483 | negative |
| 3 | 790 | 2.897627 | negative |
| 4 | 620 | 2.792392 | negative |
| 5 | 1120 | 3.049218 | negative |
| 6 | 790 | 2.897627 | one (1) colony growing |
| 7 | 5200 | 3.716003 | negative |
| 8 | 1360 | 3.133539 | negative |
| 9 | 1040 | 3.017033 | negative |

Bacteria: Listeria monocytogenes
Control

| | | | |
|---|---|---|---|
| 1 | 1720 | 3.235528 | five (5) colonies growing |
| 2 | 1840 | 3.264818 | six (6) colonies growing |
| 3 | 1440 | 3.158362 | negative (no growth) |
| 4 | 1820 | 3.260071 | five (5) colonies growing |
| 5 | 1440 | 3.158362 | one (1) colony growing |
| 6 | 1880 | 3.274158 | negative |
| 7 | 1720 | 3.235528 | negative |
| 8 | 1720 | 3.235528 | negative |
| 9 | 1740 | 3.240549 | negative |

Bacteria: Shewanella putrefaciens
Control

| | | | |
|---|---|---|---|
| 1 | 50 | 1.698970 | negative (no growth) |
| 2 | 50 | 1.698970 | negative |
| 3 | 60 | 1.778151 | negative |
| 4 | 20 | 1.301030 | negative |
| 5 | 50 | 1.698970 | negative |
| 6 | 70 | 1.845098 | negative |
| 7 | 80 | 1.903090 | negative |
| 8 | 20 | 1.301030 | negative |
| 9 | 30 | 1.477121 | negative |

Bacteria: Escherichia coli
Control

| | | | |
|---|---|---|---|
| 1 | 15100000 | 7.178977 | 460 colonies growing |
| 2 | 12900000 | 7.110590 | negative (no growth) |
| 3 | 13300000 | 7.123852 | 32 colonies growing |
| 4 | 12200000 | 7.086360 | 1170 colonies growing |
| 5 | 13400000 | 7.127105 | 4700 colonies growing |
| 6 | 12200000 | 7.086360 | 57 colonies growing |
| 7 | 14200000 | 7.152288 | 900 colonies growing |
| 8 | 13600000 | 7.133539 | 410 colonies growing |
| 9 | 7600000 | 6.880814 | 37 colonies growing |

Referring now to FIG. 1, the graph shows the effect of PHB0020 on pathogenic and spoilage bacteria identified in the table above. The graph is divided in two sections, on the left is the control showing the logarithm of colony forming units for each bacterium and on the right is the graph of colony forming units after each bacterium is exposed for 2 minutes to scalder water treated with PHB0020. The graph shows that Listeria, a gram-positive bacterium, is hard to kill and E coli, a very prolific bacterium, has the highest reduction after a 2 minute exposure.

In FIG. 2, the graph shows the logarithm of the reduction of bacterial levels for each bacterium. In most cases the log of colony forming units is less than three, with the most prolific bacterium, E coli having a log of less than five.

Thus, PHB0020 functions as an antimicrobial agent, disinfectant, or sanitizer and is extremely effective for eliminating populations of pathogenic, indicator and spoilage bacteria in commercial scalder water under industrial scalding conditions. PHB0020 is an effective means for controlling bacteria in scalder water and may be used for controlling cross-contamination during scalding. Disinfection of poultry scalder water is crucial because it is the first area within the plant in which birds are immersed in a common bath wherein bacteria can be transferred from bird to bird.

The efficacy of PHB0020 as an antimicrobial agent additive and active ingredient in an oral health care composition and method for treating mouth sores and canker sores and for reducing oral cavity, food and drink malodor is described in greater detail below.

In Examples 2-5, certain food, drink and tobacco are selected because they are known to cause oral malodor that lasts up to 72 hours after ingestion. These substances are absorbed by the intestine, metabolized in the liver, released into the bloodstream, and excreted through the lungs and other routes, hence the odor of these foods coming from the mouth. If the malodor smells like the ingested food, drink or tobacco product it can be stated with confidence that the ingested substance is the source of the malodor. Usually the malodor will disappear once the ingested substance has left the stomach. However, when PHB0020 is used in the following range, the resulting oral health care composition can immediately begin to reduce the malodor from an ingested substance.

Use Levels in Parts Per Million (ppm):

| Application for PHB0020: | Range | Target |
|---|---|---|
| Breath freshener | 1 mg/l to 2 mg/l | 1.1 mg/l |
| Oral cavity sores/lesions | 1 mg/l to 2 mg/l | 1.6 mg/l |

The composition of the present invention, hereinafter referred to as Close Call, was prepared at room temperature; using the following formulation in Table II:

TABLE II

| Ingredient | Percentage |
|---|---|
| PHB0020 | 0.0019 |
| Citric Acid | 0.0650 |
| Ascorbic Acid | 0.4000 |
| Sodium benzoate | 0.2800 |
| Potassium | 0.0160 |
| benzoate | |
| Ginger | 0.4000 |
| Xylitol | 8.3333 |
| Vitamin B₆ | 0.0490 |
| Polyglycol | 0.3380 |
| Sorbitol | 0.6500 |
| Water | 89.4701 |
| Total | 100.0000 |

The composition of the present invention is prepared by first mixing PHB0020 which contains metallic ions with deionized water and then adding in sequence the edible inorganic acids: citric acid and ascorbic acid; the alkali-metal benzoate compounds: sodium benzoate and potassium benzoate; ginger; xylitol; and Vitamin B6. The ingredients are mixed in the percentages given in Table II above. The mixture is thoroughly stirred until the metallic ions in the PHB0020 starting material are completely blended and uniformly suspended.

A placebo or control was prepared at room temperature, and is a mixture of citric acid, ascorbic acid, sodium benzoate, potassium benzoate, ginger, xylitol, Vitamin B₆, and water in the approximately the same proportions as Close Call; however, the control did not contain PHB0020.

Statistical analysis using one way analysis of variance revealed that Close Call and the placebo groups were equivalent prior to starting the study, i.e., at baseline, designated as A1 and A2 on the charts below. At the beginning of the study and after ingesting or consuming the food, drink or tobacco products under investigation and after a three-minute incubation period with the mouth closed, a reading is taken at time equals 0 (T/0). Subsequent readings on the Halimeter were taken after 5 minutes, after 30 minutes and after 60 minutes for all subjects involved in the study.

Offensive odors emanating from the oral cavity are the odiferous and “foul-smelling” compounds referred to as Volatile Sulfur Compounds (VSCs). VSCs are volatile because they are unstable and emit or release strong foul, putrid or fetid odors which are perceived as bad breath. The food, drink and tobacco products under investigation, greatly contribute to an increase in VSCs resulting in bad breath.

The Halimeter®, a portable sulfide meter manufactured by Interscan, Inc., of Chatsworth, Calif. was used to measure volatile sulfur compounds by detecting the amount of sulfur-producing bacteria in the mouth. The portable sulfide meter uses an electrochemical, voltametric sensor which generates a signal when it is exposed to sulfide and mercaptan gases and measures the concentration of hydrogen sulfide gas in parts per billion.

### EXAMPLE 2

### Garlic Study

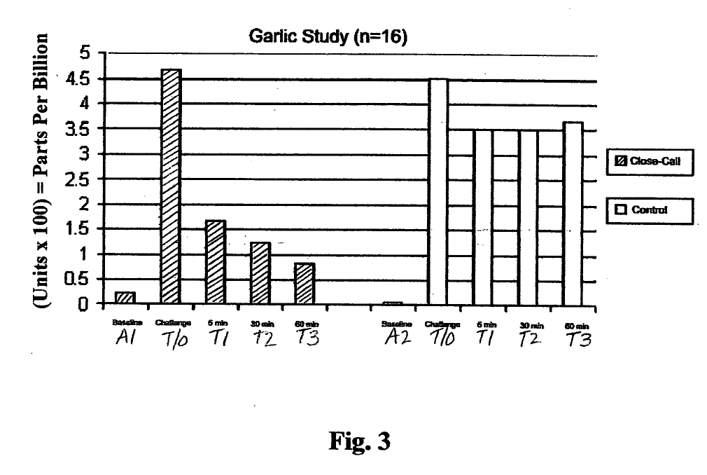

Sixteen subjects were each given one (1) peeled, raw garlic clove to eat. After a three minute incubation period, wherein each subject kept his or her mouth closed, the first reading was taken at time equals zero (T/0). After the reading at T/0, eight subjects were given Close Call with instructions to thoroughly rinse the mouth cavity and then swallow the Close Call liquid; the other eight subjects were given the placebo, without PHB0020, and were instructed to thoroughly rinse the mouth cavity, and then swallow the placebo. The reduction in breath odors as assessed by the Halimeter® organoleptic test is shown in FIG. 3.

In FIG. 3, the baseline reading A1 and A2 of the Halimeter® sensor is taken for sixteen subjects with clean, fresh breath; the Halimeter® readings are approximately 20 ppb for A1 and approximately 5 ppb for A2. The threshold for objectionable breath begins in a range from approximately 150 ppb to approximately 180 ppb. FIG. 3 shows the measurement at T/0 after the ingestion of garlic and incubation of garlic odor in the mouth is approximately 470 ppb for the subjects given Close Call and approximately 450 ppb for the subjects given the placebo. After 5 minutes, 30 minutes and 60 minutes the Halimeter® readings for Close Call users are approximately 170 ppb, approximately 125 ppb and approximately 75 ppb, respectively. For the subjects given the placebo, after 5 minutes, 30 minutes and 60 minutes, the Halimeter® readings were approximately 350 ppb, approximately 350 ppb and approximately 370 ppb, respectively. Thus, the placebo was not effective in eliminating objectionable breath odor, whereas, Close Call, the product of the present invention effectively reduced objectionable garlic odor in 30 minutes and continued to be more effective with the passage of time.

In the organoleptic assessments of garlic odor, statistically significant reductions were seen in the use of Close Call, as compared to the placebo at 5 minutes p=0.019, at 30 minutes (p<0.05) and at 60 minutes (p<0.001) as shown in FIG. 3.

### EXAMPLE 3

### Onion Study

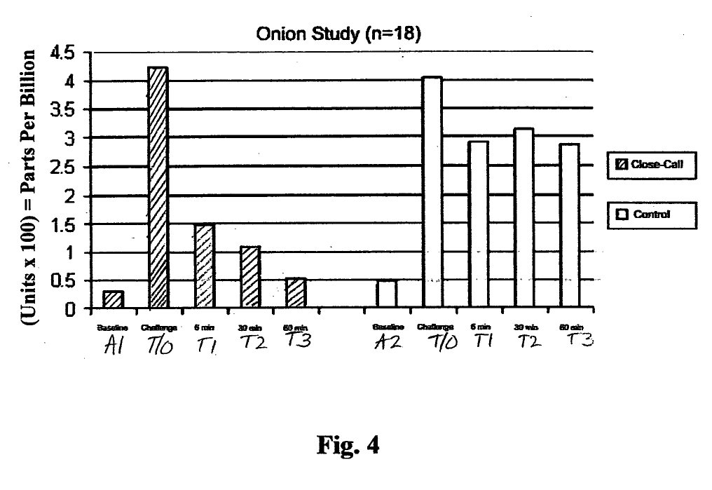

Eighteen subjects were given one (1) teaspoon of peeled, chopped raw onion to eat. After eating the onion, all subjects were given instructions to keep their mouth closed for three minutes to allow for the incubation of the onion odor. After the three minute incubation period, nine subjects were given instructions to thoroughly rinse the mouth cavity and then swallow the Close Call liquid; nine subjects were given the placebo, without PHB0020, and were instructed to thoroughly rinse the mouth cavity, and then swallow the placebo. The reduction in breath odors as assessed by the organoleptic test is shown in FIG. 4.

Each subject began the study with clean, fresh breath. The baseline reading A1 and A2 of the Halimeter® sensor are approximately 30 ppb for A1 and approximately 50 ppb for A2. The threshold for objectionable breath begins in a range from approximately 150 ppb to approximately 180 ppb. FIG. 4 shows the measurement at T/0 after the ingestion of onion and incubation of onion odor in the mouth is approximately 420 ppb for the subjects given Close Call and approximately 405 ppb for the subjects given the placebo. After 5 minutes, 30 minutes and 60 minutes the Halimeter® readings for Close Call users are approximately 150 ppb, approximately 105 ppb and approximately 50 ppb, respectively. For the subjects given the placebo, after 5 minutes, 30 minutes and 60 minutes, the Halimeter® readings were approximately 295 ppb, approximately 305 ppb and approximately 340 ppb, respectively. Thus, the placebo was not effective in eliminating objectionable breath odor, whereas, Close Call, the product of the present invention effectively reduced objectionable onion odor in 30 minutes and continued to be more effective with the passage of time.

In organoleptic assessments of onion odor, statistically significant reductions are measured in the Close Call users as compared to the placebo users at 5 minutes (p=0.05), at 30 minutes (p<0.05) and at 60 minutes (p<0.001) according to FIG. 4.

### EXAMPLE 4

### Coffee Study

Coffee with or without caffeine contains high levels of acids. These acids cause the bacteria to reproduce more quickly and create a bitter or foul taste for many people. Many other acidic type foods will do the same. Thus, coffee which is usually known to have a pleasant aroma can be an unwelcome culprit when ingested. Coffee can rapidly cause leftover food particles in the mouth to become putrefied, thereby increasing malodor.

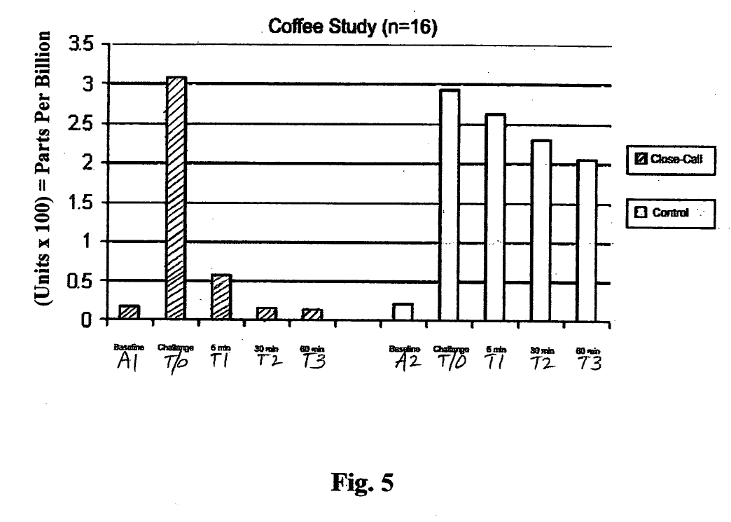

Sixteen subjects began the study with fresh, clean breath, then were given one (1) cup of coffee to drink. After drinking the coffee, and allowing a three minute incubation time, with a closed mouth, eight subjects were given Close Call with instructions to thoroughly rinse the mouth cavity and then swallow the Close Call liquid; eight subjects were given a placebo, without PHB0020, and were instructed to thoroughly rinse the mouth cavity, and then swallow the placebo. The reduction in breath odors as assessed by the Halimeter® organoleptic test is shown in FIG. 5.

The baseline readings A1 and A2, with the Halimeter® sensor are approximately 20 ppb for A1 and approximately 30 ppb for A2. The threshold for objectionable breath begins in a range from approximately 150 ppb to approximately 180 ppb. FIG. 5 shows the measurement at T/0 after the ingestion of coffee and incubation of coffee odor in the mouth is approximately 305 ppb for the subjects given Close Call and approximately 290 ppb for the subjects given the placebo. After 5 minutes, 30 minutes and 60 minutes the Halimeter® readings for Close Call users are approximately 55 ppb, approximately 20 ppb and approximately 20 ppb, respectively. For the subjects given the placebo, after 5 minutes, 30 minutes and 60 minutes, the Halimeter® readings were approximately 260 ppb, approximately 240 ppb and approximately 205 ppb, respectively. Thus, the placebo was not effective in eliminating objectionable breath odor from coffee, whereas, Close Call, the product of the present invention, effectively reduced objectionable coffee breath odor in 5 minutes and continued to be more effective with the passage of time.

When measuring volatile sulfur compounds with the Halimeter® sensor, statistically significant reductions were seen in the subjects using Close Call as compared to the placebo group at 60 minutes (p<0.004).

In organoleptic assessments of coffee odor, statistically significant reductions were seen in the Close Call users as compared to the placebo users at 5 minutes (p=0.05), at 30 minutes (p<0.05) and at 60 minutes (p<0.05). See FIG. 5.

### EXAMPLE 5

### Tobacco Study

Interestingly, for at least a century, people have smoked in order to cover up other oral odors. Cigarette smoke odor can linger in the mouth itself and mix with other smells, resulting in a particularly noxious aroma. Smoke odor comes out of two places: the mouth and the lungs. Tobacco smoke may occasionally, be detected on the breath of people who don't smoke at all. These individuals have been continually exposed to the smoke of others, and end up having telltale odor as a result.

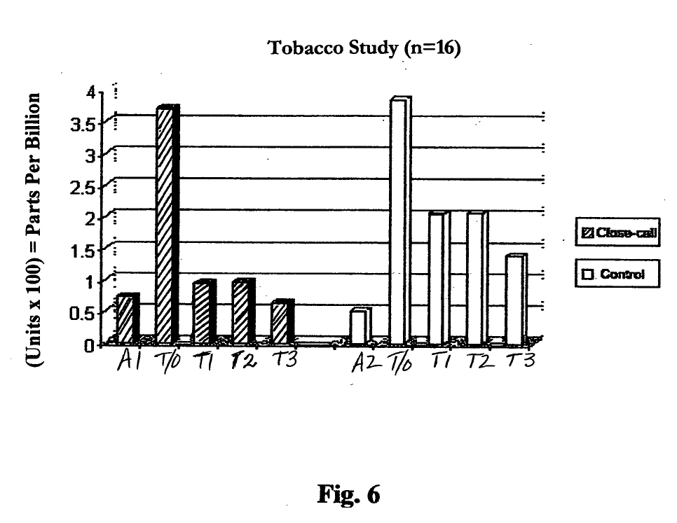

For the tobacco study, subjects were instructed to smoke one cigarette and keep the mouth closed for three minutes after smoking the cigarette. Eight subjects were given Close Call with instructions to thoroughly rinse the mouth cavity and then swallow the Close Call liquid. Eight subjects were given a placebo, without PHB0020, and were instructed to thoroughly rinse the mouth cavity, and then swallow the placebo. The test results as assessed by the Halimeter® organoleptic test are shown in FIG. 6.

Sixteen subjects began the study with clean, fresh breath and then, were given one (1) cigarette to smoke. In FIG. 6, the baseline reading A1 and A2 of the Halimeter® sensor are approximately 52 ppb for A1 and approximately 49 ppb for A2. The threshold for objectionable breath begins in a range from approximately 150 ppb to approximately 180 ppb. FIG. 6 shows the measurement at T/0 after the smoking of one cigarette and incubation of cigarette smoke odor in the mouth is approximately 360 ppb for the subjects given Close Call and approximately 370 ppb for the subjects given the placebo. After 5 minutes, 30 minutes and 60 minutes the Halimeter® readings for Close Call users are approximately 90 ppb, approximately 90 ppb and approximately 50 ppb, respectively. For the subjects given the placebo, after 5 minutes, 30 minutes and 60 minutes, the Halimeter® readings were approximately 200 ppb, approximately 200 ppb and approximately 140 ppb, respectively. Thus, the placebo was not effective in eliminating objectionable breath odor in 30 minutes, whereas, Close Call, the product of the present invention effectively reduced objectionable cigarette smoke odor in 5 minutes and continued to be more effective with the passage of time.

### EXAMPLE 6

### Handling Raw Onions and Garlic

Further testing of the novel composition, Close Call, involved people who were peeling, slicing and cutting raw odiferous vegetables, such as onions and garlic. When peeling and slicing the onions and garlic, all contact surfaces, including but not limited to, hands, countertops, cutlery, cutting boards, and the like became contaminated with the telltale odor of onions and garlic. A dilute solution of Close Call comprising 50% Close Call, without xylitol, and 50% water, was used to wash, rinse or wipe the contacted surface. Within less than approximately five (5) minutes, the telltale odor was not detectable by the persons involved in the testing.

### EXAMPLE 7

### Mouth Sores and Canker Sores

Close Call is useful as an oral health car drink that counteracts canker sores, which are small white or gray based ulcers with a red border in the mouth. These types of ulcers are generally not contagious. Fatigue, stress or allergies can increase the likelihood of these types of ulcers. Other mouth sores can be caused by bacterial, fungal, or viral infections, broken skin inside the mouth, immune disorders, nutritional deficiency and an excel of dietary nitrates and nitrites found in bacon, ham, processed meats, vegetables and other foods.

Twelve subjects between the ages of 10 to 20 years, with canker and mouth sores were given daily doses of Close Call. A dose consists of approximately 2 fluid ounces. Personal reports confirm that within approximately one (1) hour of taking the Close Call dosage, the ulcerated sores were less painful and beginning to heal. In less than approximately three (3) days, there were no ulcerated areas in the mouth of any subject.

Close Call is an effective product because of the powerful antibacterial ingredient PHB0020 and the use of Vitamin B6 (pyridoxine) and Vitamin C (ascorbic acid) that counter recurrent canker sores. With regard to mouth sores, Vitamin C is effective in killing viruses and inhibiting the formation of nitrosamines.

PHB0020 allows metallic ions to be carried and held in suspension and made available to organisms in a variety of pH values with no change to the metallic ions, such as copper ions. Copper is recognized as a nutrient required by the human body and the Close Call formula uses one (1) milligram (mg) of copper per liter (1) of product. It is understood that other metallic ions, such as zinc and silver can also be uniformly suspended when mixed with the PHB0020 solution.

The oral care composition of the present invention can be formulated into a range of oral care products including, but not limited to, pastes, gels, mouthwashes, lozenges, chewing gums, dental floss, orally consumable film and mouth spray; the preferred formulation is as a liquid rinse.

The method of treating or preventing malodors or disease conditions of the oral cavity in warm-blooded animals, including humans, is by applying an oral care effective amount of the oral care composition of the present invention to the oral cavity. The oral care effective amount of the oral care composition of the present invention is preferably applied to the mucosal tissue of the oral cavity, to the gingival tissues of the oral cavity, and/or surface of the teeth, for the treatment or prevention of the above-mentioned disease or conditions of the oral cavity in one or more conventional ways. For example, the gingival or mucosal tissue may be rinsed with a solution, such as a rinse containing the composition of the present invention. If in the form of a paste, gel or lozenge that can be liquefied in the mouth or the gingival/mucosal tissue or teeth may be bathed in the liquid and/or lather generated by brushing the teeth, for a sufficient time, preferably from about 10 seconds to 10 minutes, more preferably from about 30 seconds to 60 seconds.

The method of the present invention generally further involves swallowing most of the composition following such contact as liquefying, rinsing, gargling, or brushing.

The novel composition of the present invention is effective against various organisms, diseases and malodors and can create a healthy environment inside the mouth of warm-blooded animals, including humans. The composition of the present invention provides users with a clean, fresh breath, heals and prevents mouth sores and canker sores and clears the way to lives filled with hugs and kisses.

While the invention has been described, disclosed, illustrated and shown in various terms of certain embodiments or modifications which it has presumed in practice, the scope of the invention is not intended to be, nor should it be deemed to be, limited thereby and such other modifications or embodiments as may be suggested by the teachings herein are particularly reserved especially as they fall within the (breath) breadth and scope of the claims here appended.

We claim:

1. An oral care composition comprising an oral care effective amount of:
(a) a mixture comprising PHB0020 containing metallic ions, edible inorganic acid, at least one alkali benzoate compound, ginger, polyglycol, sorbitol, pyridoxine (vitamin B6); and
(b) water to form a solution for use in the oral cavity of warm-blooded animals.

2. The oral care composition of claim 1, further comprising a sweetener.

3. The oral care composition of claim 2, wherein the sweetener is xylitol.

4. The composition of claim 1, wherein the metallic ions are selected from the group consisting of copper, silver and zinc.

5. The composition of claim 4, wherein the metallic ions are copper ions.

6. The composition of claim 1, wherein the edible inorganic acid is at least one of citric acid, ascorbic acid and phosphoric acid.

7. The composition of claim 6, wherein the edible inorganic acid is a combination of citric acid and ascorbic acid.

8. The composition of claim 1, wherein the alkali-metal benzoate compound is selected from the group consisting of sodium benzoate, potassium benzoate and calcium benzoate.

9. The composition of claim 8, wherein the alkali-metal benzoate compound is a combination of sodium benzoate and potassium benzoate.

10. A method of preparing an oral care composition that is useful for reducing malodors from food, tobacco and beverage products, comprising the steps of:
(a) mixing PHB0020 containing metallic ions with deionized water;
(b) adding an edible inorganic acid, at least one alkali benzoate compound, ginger, poly glycol, sorbitol, xylitol, pyridoxine (vitamin B6); and
(c) stirring until completely blended and metallic ions are uniformly suspended.

11. The method of claim 10, wherein the metallic ions are selected from the group consisting of copper, silver and zinc.

12. The method of claim 11, wherein the metallic ions are copper ions.

13. The method of claim 10, wherein the edible inorganic acid is at least one of citric acid, ascorbic acid and phosphoric acid.

14. The method of claim 13, wherein the edible inorganic acid is a combination of citric acid and ascorbic acid.

15. The method of claim 10, wherein the alkali-metal benzoate compound is selected from the group consisting of sodium benzoate, potassium benzoate and calcium benzoate.

16. The method of claim 15, wherein the alkali-metal benzoate compound is a combination of sodium benzoate and potassium benzoate.

17. A method for treating or preventing ulcerated conditions of the oral cavity in warm-blooded animals, including humans, comprising applying an oral care effective amount of the composition of claim 1 to the oral cavity.

18. The method of claim 17 comprising applying the composition of claim 1 in a form selected from the group consisting of paste, gels, mouth sprays and lozenges that can be liquefied in the mouth, rinsed and swallowed.

19. A method for treating or preventing oral cavity malodors resulting from the ingestion of substances selected from the group consisting of onion, garlic, coffee, and tobacco comprising applying an oral care effective amount of the composition of claim 3 to the oral cavity.

20. The method of claim 19 further comprising applying the composition of claim 3 in a form selected from the group consisting of paste, gels, mouth sprays and lozenges that can be liquefied, rinsed and swallowed.

21. A composition for removing the malodor of peeled, odoriferous raw vegetables comprising a dilute solution of the composition of claim 1 which can be applied to a surface that is contacted by peeled, odoriferous raw vegetables.

22. The composition of claim 21, wherein the malodors are removed from the surface selected from the group consisting of hands, counter tops, cutlery, clothing and cutting boards.
